(19) United States
(12) Patent Application Publication
(10) Pub. No.: US 2006/0253167 A1
(43) Pub. Date: Nov. 9, 2006

(54) Device, method and stimulus unit for testing neuromuscular function

(76) Inventors: Ronald L. Kurtz, Oakville (CA); John R. Mumford, Mississauga (CA)

Correspondence Address: BERESKIN AND PARR, 40 KING STREET WEST, BOX 401, TORONTO, ON, M5H 3Y2, CA

(21) Appl. No.: 11/407,296

(22) Filed: Apr. 20, 2006

Related U.S. Application Data
(60) Provisional application No. 60/672,853, filed on Apr. 20, 2005.
(60) Provisional application No. 60/774,646, filed on Feb. 21, 2006.

(51) Int. Cl. A61N 1/18

(52) U.S. Cl. 607/48

(57) ABSTRACT

Embodiments relate generally to devices, methods and stimulus units for use in measuring neuromuscular function. One particular embodiment relates to a device comprising a substrate, wherein the substrate has a base portion and at least one limb coupled to the base portion. The device further comprises at least two stimulation electrodes operably associated with the substrate for providing a stimulus to a body part. The device further comprises at least two sensing electrodes operably associated with the substrate for sensing an electrical potential in the body part. The at least two sensing electrodes are spaced from the at least two stimulation electrodes. The device further comprises an elongate member coupled to one of the base portion and the at least one limb and having indicia for indicating separation of the at least two stimulation electrodes and the at least two sensing electrodes.

### CROSS-REFERENCE TO RELATED APPLICATIONS

This application claims the benefit of U.S. Provisional Patent Application No. 60/672,853, filed Apr. 20, 2005 and U.S. Provisional Patent Application No. 60/774,646, filed Feb. 21, 2006, the entire contents of both of which are hereby incorporated by reference.

### TECHNICAL FIELD

Embodiments relate to a device, method and stimulus unit for testing neuromuscular function. In particular, embodiments employ one or more stimulation and sensing electrodes disposed on a substrate for placement on a body part of a test subject.

### BACKGROUND

When performing nerve conduction testing, such as testing for carpal tunnel syndrome, for example, a series of stimuli are provided to a part of the body adjacent to the nerve desired to be tested and the response of the body to each stimulus is measured. Such responses may include a muscle response, in the form of a compound muscle action potential (CMAP), and a nerve response, in the form of a sensory nerve action potential (SNAP).

A relevant parameter in determining whether a subject may be experiencing carpal tunnel syndrome, or other forms of systemic or entrapment neuropathies, is the nerve conduction velocity of the stimulated nerve. Nerve conduction velocity is determined by measuring the distance between the stimulation site and detection site on the stimulated body part and then observing the time elapsed between stimulus of the nerve and detection of the SNAP evoked in response to the stimulus.

Typically, a medical technologist performing the nerve conduction testing will take a measuring tape and place it along the body part to estimate the distance between the stimulating electrode and the sensing electrode, once the electrodes are in place. Alternatively, the technologist may measure a fixed distance and then place the stimulating and sensing electrodes accordingly. Such manual measurement methods are prone to error and can be cumbersome, requiring the physician to locate the measuring tape and position it against the subjects body, while attempting to keep the patient still, in order to take the distance measurement.

It is desired to address or ameliorate one or more of the shortcomings or disadvantages of existing nerve conduction testing techniques, equipment or arrangements, or to at least provide a useful alternative thereto.

### SUMMARY

Embodiments relate generally to devices, methods and stimulus units for use in measuring neuromuscular function. One particular embodiment relates to a device comprising a substrate, wherein the substrate has a base portion and at least one limb coupled to the base portion. The device further comprises at least two stimulation electrodes operably associated with the substrate for providing a stimulus to a body part. The device further comprises at least two sensing electrodes operably associated with the substrate for sensing an electrical potential in the body part. The at least two sensing electrodes are spaced from the at least two stimulation electrodes. The device further comprises an elongate member coupled to one of the base portion and the at least one limb and having indicia for indicating separation of the at least two stimulation electrodes and the at least two sensing electrodes.

The indicia may be selected from the group consisting of: magnetic indicia; electrical indicia; optical indicia; and mechanical indicia. The substrate may be flexible to conform to a shape of the body part when positioned over the body part. The body part may be a hand and wrist area of an arm. Alternatively, the body part may be a leg and ankle area.

The stimulation and sensing electrodes may each have a layer of conductive gel disposed on portions thereof. The stimulation and sensing electrodes may be covered by a protective material wherein, in use of the device, the protection material is removed before placement of the stimulation and sensing electrodes on the body part. The at least two sensing electrodes may be positioned distal to at least two stimulating electrodes along the body part. The at least two sensing electrodes may be positioned proximal to at least two stimulating electrodes along the body part.

The at least two sensing electrodes may comprise a first electrode pair, wherein one of the sensing electrodes in the pair is an active electrode, and the other of the sensing electrodes in the pair is a reference electrode. The device may further comprises a scanner for reading the indicia on the distance measurement member. The substrate and/or the distance measurement member may comprise a unique identifier of the device. The unique identifier may be encoded on one of the distance measurement member and the substrate. The unique identifier may be machine-readable.

The device may further comprise a coupling unit for electrically coupling a current stimulation controller to the at least two stimulating electrodes. The coupling unit may be supportingly connectable to the base portion of the substrate. The coupling unit may be connectable to the base portion by conductive connectors. The coupling unit may comprise a temperature sensor, which may comprise am infrared optical sensor.

Another embodiment relates to a stimulus unit for use in measuring neuromuscular function, comprising: a substrate, the substrate having a base portion and at least one limb coupled to the base portion; at least two stimulation electrodes operably associated with the substrate for providing a stimulus to a body part; and at least two sensing electrodes operably associated with the substrate for sensing an electrical potential of the body part, wherein the at least two sensing electrodes are spaced from the at least two stimulation electrodes and wherein the at least one limb has at least one extensible section for permitting adjustment of the spacing between the at least two sensing electrodes and the at least two stimulation electrodes.

Another embodiment relates to a device for use in measuring neuromuscular function, comprising: a substrate, the substrate having a base portion and at least one limb coupled to the base portion; at least two stimulation electrodes operably associated with the substrate for providing a stimulus to a body part; at least two sensing electrodes operably associated with the substrate for sensing an electrical potential in the body part in response to the stimulus, wherein the at least two sensing electrodes are spaced from the at least two stimulation electrodes; and distance measurement means fixed relative to the substrate for measuring separation of the at least two stimulation electrodes and the at least two sensing electrodes.

Another embodiment relates to a stimulus unit for use in measuring neuromuscular function, comprising: a substrate, the substrate having a base portion and two limbs coupled to the base portion; at least two stimulation electrodes operably associated with the base portion for providing a stimulus to a body part; and at least one sensing electrode operably associated with each limb for sensing an electrical potential in the body part in response to the stimulus, wherein the at least two stimulation electrodes are spaced from the sensing electrodes.

Another embodiment relates to a method of measuring a separation between at least one distal electrode and at least one proximal electrode, the at least one distal electrode having an elongate member fixed relative to the at least one distal electrode, indicia being formed on or in the elongate member, the method comprising: affixing the at least one proximal electrode to a proximal part of a body; affixing the at least one distal electrode to a distal part of the body; securing a scanner relative to the at least one proximal electrode; and drawing a free end of the elongate member past the scanner to expose the indicia to the scanner and thereby measure the separation between the at least one distal electrode and the at least one proximal electrode.

Another embodiment relates to a method of measuring a separation between at least one distal electrode and at least one proximal electrode, the at least one distal electrode being coupled to the at least one proximal electrode using an extensible electromechanical distance measurement sensor, the method comprising: affixing the at least one proximal electrode to a proximal part of a body; extending the at least one distal electrode relative to the at least one proximal electrode by extending the electromechanical distance measurement sensor; and determining the separation based on an output of the extended electromechanical distance measurement.

### BRIEF DESCRIPTION OF THE DRAWINGS

Embodiments are described below in further detail, by way of example only, with reference to the accompanying drawings, in which:

FIG. 1 is a block diagram of a system for automatic nerve conduction testing, according to one embodiment;

FIG. 2 is a diagram illustrating connection of a coupling unit to a stimulus unit when the stimulus unit is attached to a wrist and hand area on an arm;

FIG. 3 is a perspective view of the coupling unit and stimulus unit of FIG. 2, shown connected and showing insertion of a distance measurement member into the coupling unit;

FIG. 4 is a schematic representation of a stimulus unit according to one embodiment;

FIG. 5 is a schematic representation of a stimulus unit according to another embodiment;

FIG. 6 is a schematic representation of a stimulus unit according to another embodiment;

FIG. 7 is a schematic representation of a stimulus unit according to another embodiment;

FIG. 8A is a plan view of a stimulus unit according to another embodiment;

FIG. 8B is a bottom view of the stimulus unit of FIG. 8A;

FIG. 9A is a plan view of a stimulus unit according to another embodiment;

FIG. 9B is a bottom view of the stimulus unit of FIG. 9A;

FIG. 10A is a plan view of a stimulus unit according to another embodiment;

FIG. 10B is a bottom view of the stimulus unit of FIG. 10A;

FIG. 11 is an illustration of a distance measurement member according to one embodiment;

FIG. 12 is an illustration of a distance measurement member according to another embodiment;

FIG. 13 is an illustration of a distance measurement member according to another embodiment;

FIG. 14 is a flow chart of a method of nerve conduction testing; and

FIG. 15 is an example cross-section of a base portion of a stimulus unit.

### DETAILED DESCRIPTION

Embodiments of the invention can be used to apply an automatic nerve conduction test for systemic or entrapment neuropathies, such as Carpal Tunnel Syndrome. During the test, a series of impulse stimuli are applied to a subject's body part adjacent a nerve or nerve group. The responses to the stimuli are analyzed to detect the evoked action potentials (for example, CMAP for a motor nerve test and SNAP for a sensory nerve test), and to measure the onset latency and peak amplitude of the responses. Other measureable parameters of interest include peak latency, duration and the integrated area under the response curve between onset and the peak amplitude.

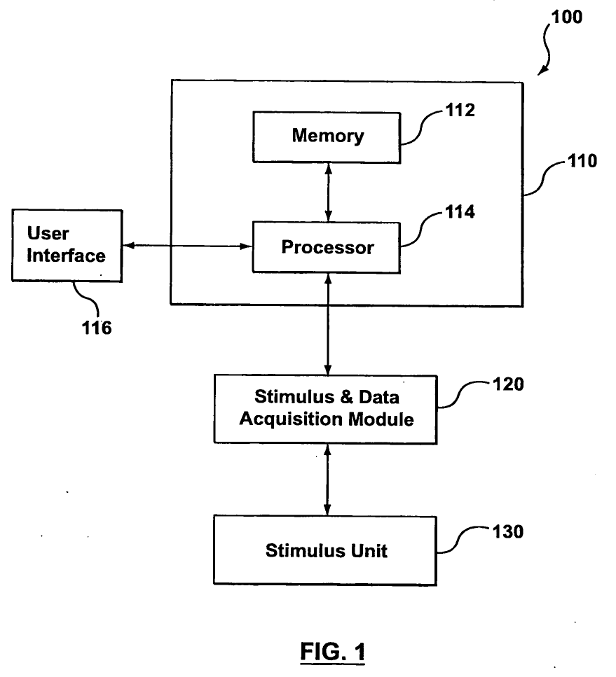

Referring to FIG. 1, there is shown a system 100 for performing automatic nerve conduction testing. System 100 comprises a control module 110 that interfaces with a stimulus unit 130 via a stimulus and data acquisition module 120 to provide stimuli to a body part and detect evoked responses, such as CMAP and SNAP responses, to the stimuli. Other evoked responses that may be detected inculde F-wave, A-wave and H-reflex responses. Control module 110 may be in the form of a computer device, such as a laptop, desktop personal computer or a handheld computing device.

Control module 110 comprises a processor 114 and memory 112. Control module 110 has a user interface 116 associated therewith that communicates with processor 114 to enable a user to interface with system 100 during, before or after the testing. The memory 112 stores computer program instructions for execution by processor 114 during performance of the automatic nerve conduction testing. Memory 112 also stores a first-in-first-out stack of sampled response waveforms (traces) for analysis by processor 114. Processor 114 controls stimulus and data acquisition module 120, which in turn controls the output of stimulus unit 130.

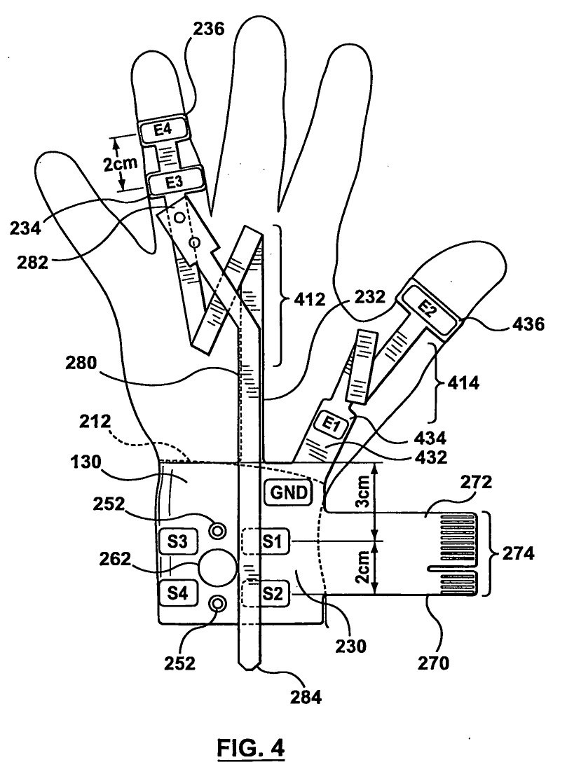

Stimulus unit 130 has two or more stimulus electrodes (for example, S1, S2, S3 and S4 shown in FIG. 4) for contacting the skin of the body adjacent a nerve that is desired to be tested and also has two or more sensing electrodes (for example, E1, E2, E3 and E4 shown in FIG. 4) for sensing the action potentials, such as CMAP and SNAP, on the skin at body part locations spaced from the stimulus sites. According to one embodiment, the stimulus unit 130 may be used to stimulate more than one nerve grouping at the same time. For example, stimulus unit 130 may be used to stimulate the median and ulnar nerve groupings in the hand simultaneously and separately detect the responses to that stimulation. Alternatively, stimulus unit 130 may detect responses to stimulus of only a single nerve grouping. Examples of embodiments of stimulus unit 130 are shown in FIGS. 4 to 7, 8A, 8B, 9A, 9B, 10A and 10B.

Stimulus and data acquisition module 120 has one or more controllers (not shown) for receiving and interpreting commands from processor 114, for conditioning response signals received from stimulus unit 130 and providing such conditioned response signals to processor 114 for analysis according to the stored computer program instructions in memory 112. Example commands received at stimulus and data acquisition module 120 from processor 114 include stimulus intensity setting commands and operational commands, such as start or stop commands. Additionally, if stimulus unit 130 is configured to provide (or cooperate with stimulus and data acquisition module 120 to provide) a temperature measurement or a measurement of the distance between the stimulation and detection points, such measurements may be provided by stimulus and data acquisition module 120 to processor 114 in response to an appropriate command received at stimulus and data acquisition module 120.

The task of processor 114 is to establish the neuromuscular function testing protocol to be administered via stimulus unit 130 and to analyze each stimulus-response waveform passed from the signal detection and processing framework (i.e. stimulus unit 130 and stimulus and data acquisition module 120).

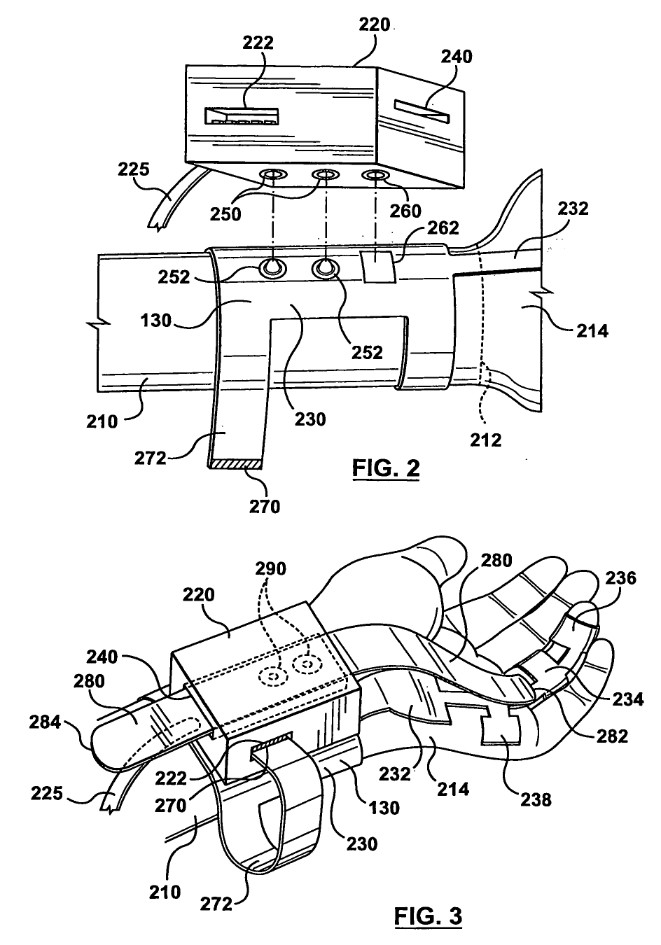

Referring also to FIGS. 2 and 3, stimulus unit 130 is shown in further detail, in use on a wrist and hand area of a person's arm. Stimulus unit 130 connects electrically with stimulus and data acquisition module 120 via a coupling unit 220, which couples directly to stimulus unit 130 to provide a stimulus current and to receive the evoked action potentials in response.

Coupling unit 220 forms part of stimulus and data acquisition module 120. Coupling unit 220 may be a dumb unit, in that it does not contain a controller exercising specific control functions, in which case another part of an underside stimulus and data acquisition module 120 located away from coupling unit 220, and in communication therewith via cable 225, performs the stimulation control and signal processing functions. Alternatively, coupling unit 220 may include a controller for performing stimulus control and/or received signal processing functions.

Coupling unit 220 couples to stimulus unit 130 by one or more connectors to position coupling unit 220 in a fixed location relative to stimulus unit 130. The connectors shown in FIG. 2 are snap connectors, with receiving parts 250 located on an underside of coupling unit 220 and projecting parts 252 located on an upper surface of stimulus unit 130. These connecting parts may be formed of conductive material, such as a conductive metal, for enabling a current stimulus to be provided from coupling unit 220 to stimulus unit 130 via the one or more connectors. Example conductive metals include nickel-plated brass or stainless steel. Instead of snap connectors, other forms of conductive connector may be employed.

In one embodiment, snap connector parts 250, 252 are not used for providing current stimulus signals, but are instead used to close a circuit (with a conductor extending between the two projecting parts 252) to provide an indication to stimulus and data acquisition module 120 that coupling unit 220 is connected to stimulus unit 130. In a further alternative embodiment, one or more non-conductive connecting parts may be used to form a connector connecting coupling unit 220 to stimulus unit 130.

Stimulus unit 130 has an output connector 270 located on an end of a connector limb 272 for providing evoked response signals detected by the one or more sensing electrodes to stimulus and data acquisition module 120, via coupling unit 220. Output connector 270 is releasably received in a socket 222 formed in coupling unit 220. Socket 222 has a structure formed for receipt of output connector 270 and for forming electrical connections with each of the conductors (which are, in turn, connected to the sensing electrodes) along connector limb 272. Connector limb 272 resembles a flexible ribbon cable. If the current stimulus wave-forms are not provided to stimulus unit 130 by the physical connection of connecting parts 250, 252, then they may be provided by conductors connected to the stimulating electrodes via connector 270.

Stimulus unit 130 has a base portion 230, with at least one limb 232 extending therefrom, in addition to connector limb 272. Limb 232 has at least one sensing electrode positioned on the limb 232 for placement at any desired site for detection of CMAP or SNAP (or both) responses, depending on the type of testing that is to be conducted. One or more stimulus electrodes, together with a ground electrode (GND), are located in or adjacent base portion 230. Limb 232 extends distally of wrist crease 212 and crosses at least part of the palm 214. As shown in FIG. 3, limb 232 has two sensing electrodes 234, 236 located toward a distal end of limb 232. Optionally, a third sensing electrode 238 may be located more proximally on limb 232, intermediate base portion 230 and distal sensing electrodes 234, 236, for sensing a CMAP response from the hypothenar area.

Stimulus unit 130 is formed mostly of flexible materials for placement on anatomical structures and for generally conforming to the shape of such anatomical structures. For example, base portion 230 is intended to be positioned proximally of a wrist crease 212 so is to extend at least partially along and around part of a forearm 210. Certain parts of stimulus unit 130 (for example, those around the electrodes) have an adhesive substance, such as a foam adhesive layer, on a underside thereof, for affixing the stimulus unit to the relevant anatomical structures prior to testing. Flexible circuitry extends through stimulus unit 130 between the electrodes and connectors. Thus, stimulus unit 130 can be used with anatomical structures of varying shapes and sizes due to its flexibility and ability to conform and adhere to anatomical structures, as required.

Stimulus unit 130 employs a substrate of a flexible material, such as a medical grade polyester film (or other materials having similar properties). The substrate may be about 3 to 8 thousandths of an inch thick, for example. Where adhesive is required to affix a part of the stimulus unit 130 to an anatomical structure, this adhesive may be provided on a layer of medical grade adhesive foam of about 1/32 of an inch thickness. The foam is adhered to an insulation layer on the substrate on one side with a relatively strong adhesive and has an adhesive of relatively less strength for removable attachment to the test subject. The electrodes may comprise a silver or silver chloride layer formed on the substrate. The substrate also has flexible circuit tracings formed thereon for constituting the conductors between electrodes and the input and/or output connector. Such circuit tracings may comprise silver and a dielectric layer. An example of the layers of stimulus unit 130 is shown and described in further detail in relation to FIG. 15.

Prior to affixation to the body part, stimulus unit 130 may have backing sheets on those part of stimulus unit 130 that have an adhesive substance on their undersides for adhesion to the skin. Each such backing sheet is removed immediately prior to adhesion of the relevant part of stimulus unit 130 to the corresponding anatomical structures. For the sensing, stimulus and ground electrodes, an area of conductive gel, such as hydrogel, is interposed between the electrode and the skin surface (instead of the adhesive foam), for facilitating conductivity of electrical signals between the electrodes and the skin.

Stimulus unit 130 is a generally flat device, as viewed from the user's perspective, prior to affixation to the test subject. However, stimulus unit 130 does have several layers, as described above. In use of stimulus unit 130, and with the backing sheets removed, the adhesive foam parts and electrodes are positioned to lie against the skin. These skin contact surfaces may be conveniently referred to as being formed on the underside of the stimulus unit 130. Printed labeling, including affixation instructions, may be provided on the side of stimulus unit 130 that does not contact the skin.

Coupling unit 220 has a temperature sensor 260, such as an infrared temperature sensor, positioned on a lower surface of coupling unit 220 that is to be positioned to face the body part when coupled to stimulus unit 130. Temperature sensor 260 is used to detect the temperature of the skin prior to and/or during the testing. If temperature sensor 260 is used to take a temperature measurement prior to initiation of the testing, it can be placed over the palmar region or other anatomical structure, as appropriate, prior to connection of coupling unit 220 to stimulus unit 130. Alternatively, the temperature measurement may be obtained after connection of coupling unit 220 to stimulus unit 130, provided that stimulus unit 130 has an appropriate opening 262 to allow temperature sensor 260 to directly sense the skin temperature.

Coupling unit 220 also has a slot 240 formed in a housing of coupling unit 220 for receiving a distance measurement strip 280. Slot 240 extends all the way through coupling unit 220 so that the distance measurement strip 280 can be drawn though slot 240 in order to perform the distance measurement function, as described herein. In the embodiment shown in FIG. 3, scanners 290, such as optical scanners, are used to scan indicia located on distance measurement strip 280 between a free end 284 and a fixed end 282, which is attached to limb 232 in the vicinity of a sensing electrode.

Fixed end 282 may be attached to limb 232 by an adhesive or a mechanical connection, for example. Fixed end 282 may be attached to limb 232 in such a way that allows the distance measurement strip to be manually torn off or otherwise removed once it has been used.

Example distance measurement strips having different forms of indicia are shown in FIGS. 11 to 13. For the embodiment shown in FIG. 3, the indicia on distance measurement strip 280 are optically readable indicia that can be read by scanners 290 as the distance measurement strip 280 and the indicia thereon passes by the scanners 290 when distance measurement strip 280 is drawn through slot 240 in coupling unit 220.

Scanners 290 are located within the housing of coupling unit 220 and are positioned to sense indicia on the distance measurement strip 280 and to provide output signals to stimulus and data acquisition modules 120 via cable 225. The electrical signals corresponding to the scanned optical indicia are processed within stimulus and data acquisition module 120 to determine the distance between the stimulus electrodes, which are in a fixed position relative to optical scanners 290, and sensing electrodes located on a distal extremity of the body part, such as a finger, the size and length of which will depend on the physical characteristics of the test subject.

The distance measurement calculation is performed by stimulus and data acquisition module 120, taking into account the point along distance measurement strip 280 at which scanners 290 are positioned when distance measurement strip 280 comes to rest, the known distance between scanners 290 and the stimulating electrodes when coupling unit 220 is connected to stimulus unit 130 and the known distance between the point at which fixed end 282 is connected to limb 232 and the sensing electrodes 234, 236 located on limb 232.

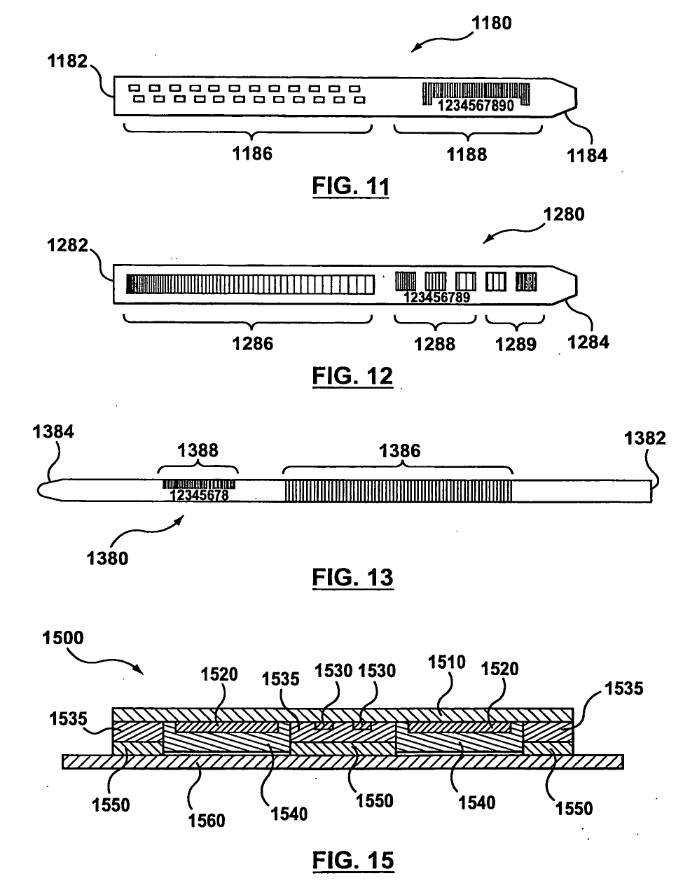

Depending on the type and/or configuration of the indicia on distance measurements strip 280, only one scanner 290 may be necessary. For example, if the indicia comprise gray scale indications, such as is shown in FIG. 12, only one optical scanner is required. However if the indicia comprised offset quadrature indicia, such as is shown in FIG. 11, two scanners are required to be able to determine the distance based on such indicia. Alternatively, the pair of quadrature scanners 290 may be offset and the indicia aligned with no offset.

In alternative embodiments, indicia other than optically readable indicia may be formed in, positioned on or otherwise fixed in relation to distance measurement strip 280 for enabling determination of the distance between the sensing electrodes and stimulation electrodes. Mechanical markings or formations may be applied to distance measurement strip 280, for example, in the form of crenulations along one edge or deformations in part of the strip. Alternatively, electrical or magnetic indicia may be formed in, or in relation to, distance measurement strip 280 for sensing by corresponding sensors in coupling unit 220. Whether the indicia is optical, mechanical, electrical, magnetic, a combination of two or more of these or any other machine-readable form, the indicia are, at least according to such embodiments, using an appropriate sensing means positioned within coupling unit 220 for generating electrical signals for transmission to a signal processor within stimulus and data acquisition module 120 via cable 225.

In other alternative embodiments, the distance measurement strip 280 may be provided with human readable indicia for alignment with a fixed visible alignment marker on coupling unit 220 or a part of base portion 230, so that a person may readily determine from the human readable indicia and the alignment marker the distance between the sensor electrodes and the stimulus electrodes. Alternatively, instead of distance measurement strip 280 being fixed at a location near the sensor electrodes and having its free end extend across base portion 230, distance measurement strip 280 may be fixed at a location on or adjacent base portion 230 and extending toward the sensing electrodes for alignment of human readable indicia on the strip with an alignment marker positioned at a particular location on limb 232 adjacent to the sensing electrodes. For such embodiments using human readable indicia, the distance measurement determined with reference to the alignment marker would need to be input into control module 110 via user interface 116.

In a further alternative embodiment using human readable indicia, coupling unit 220 may be provided with an extensible measuring strip that retractably extends from coupling unit 220 for visual comparison with an alignment marker positioned adjacent one or more of the SNAP sensing electrodes 234, 236. In an alternative of such an embodiment, the retractable strip may use machine-readable indicia to determine the distance according to indicia that can be read from the strip by a scanner within coupling unit 220 when a free end of the retractable strip is positioned at the alignment marker.

Particular embodiments of further optical distance measurement methods may include use of stereoscopic optical sensors, triangulation of a marker light (where the marker is attached at or adjacent the sensing electrodes and the optical sensor is located in the coupling unit 220) and optical pattern recognition techniques. In a further embodiment, an acoustic time-of-flight calculation may be performed in relation to a marker source attached at or adjacent the sensing electrodes, with the acoustic sensor located in the coupling unit 220. Embodiments employing electrical distance measurement may include sensing a deformation of a wire loop having a modified self-inductance depending on its position along the distance measurement strip or along an extensible section in limb 232.

Electromechanical embodiments may use transducers, such as strain gauges, potentiometers or linear variable differential transformers (LVDT). Such embodiments may use structure embedded within distance measurement strip 280 or an extensible section in limb 232 and corresponding sensing structure and circuitry within coupling unit 220. Specific mechanical distance measurement embodiments may employ a form of tape measure built into coupling unit 220, with sensors to determine the position or rotation of the tape wheel within coupling unit 220, and or human readable indicia visible on the tape as it is extended from the coupling unit 220.

In certain embodiments, stimulus unit 130 may be employed with only a simple mating connector to connect to connector 270 in place of coupling unit 220. For such an embodiment, as there is no necessity to connect coupling unit 220 to stimulus unit 130, connector projections 252 are not required. Also, without a temperature sensor 260, opening 262 in stimulus unit 130 is not required.

The embodiment of stimulus unit 130 shown in FIGS. 2 and 3 has a base portion 230, a connector limb 272 and a distally extending limb 232 connected to, and extending away from, the base portion 230. Connector limb 272 is connected to, and extends away from, a proximal part of base portion 230. The base portion 230 is used to position the stimulation electrodes adjacent the nerve bundle desired to be stimulated during the testing, while the limb 232 extends distally to position the sensing electrodes in the desired locations for sensing SNAP and/or CMAP evoked responses. The connector limb 272 is used to couple to the stimulus and data acquisition module 120 and provide output signals corresponding to the electrical signals coupled to the conductors exposed by connector 270.

The base portion 230, distally extending limb 232 and connector limb 272 or a basic configuration of the stimulus unit 130. Variations of such a basic configuration form further embodiments, as described below. For example, stimulus unit 130 may have more than one distally projecting limb 232. Further, connector limb 272 may extend from a different part of the base portion 230, depending on whether the stimulus unit is for right hand or left hand testing, for example. While the precise shape and configuration of base portion 230 may vary, the features and functions of base portion 230 according to the basic configuration described above are common to all embodiments.

Referring also now to FIG. 4, one particular embodiment of stimulus unit 130 is shown schematically, as located on a person's right hand for performing median and ulnar nerve conduction testing,

Base portion 230, as shown in FIG. 4, has two stimulation electrode pairs S1, S2 and S3, S4 formed in the substrate. The first stimulation electrode pair S1, S2 is to be positioned over the median nerve running centrally through the wrist, while the second electrode pair S3, S4 is to be positioned over the ulnar nerve. In the examples shown in FIG. 4, a distal edge of base portion 230 is approximately aligned with the wrist crease 212 and the base portion 230 is fixed in position by adhesion with the skin. In this position, a ground electrode GND is positioned distally of the stimulation electrodes but proximally of the sensing electrodes and generally toward a distal edge or area of base portion 230.

Stimulus unit 130, as shown in FIG. 4, has a first limb 232 extending distally from base portion 230 for attachment to the fourth digit (ring finger) on the right hand. Fixed end 282 of distance measurement strip 280 is affixed to limb 232 adjacent, but proximal of, sensing electrode 234. Free end 284 of distance measurement strip 280 extends proximally from fixed end 282 for passing through slot 240, when coupling unit 220 is connected to the stimulus unit 130.

Stimulus unit 130, as shown in FIG. 4, has a second limb 432 connected to, and extending distally from, base portion 230. Second limb 432 has first and second sensing electrodes E1, E2 formed in respective first and second attachment portions 434, 436 having adhesive on an underside thereof for holding the sensing electrodes E1, E2 on to the skin at desired locations. Sensing electrode E1 is positioned approximately over the middle of the thenar area, while sensing electrode E2 is wrapped around a distal joint of the thumb.

The first and second limbs 232, 432 each have a respective extensible portion 412, 414 for accommodating size differences among hands by allowing lesser of greater extension of the extensible portions 412, 414, depending on hand size. Extensible portions 412, 414 may be simply formed of a somewhat flattened coil or loop in the respective limb.

The stimulus unit 130 shown in FIG. 4 has a connector 270 with a plurality of connecting conductors 274 located at an end of connector limb 272. Connecting conductors 274 communicate with conductors formed in the substrate of stimulus unit 130 and extending through the limbs 232, 432 and base portion 230. Connecting conductors 274 connect with corresponding conductors in socket 222 of coupling unit 220.

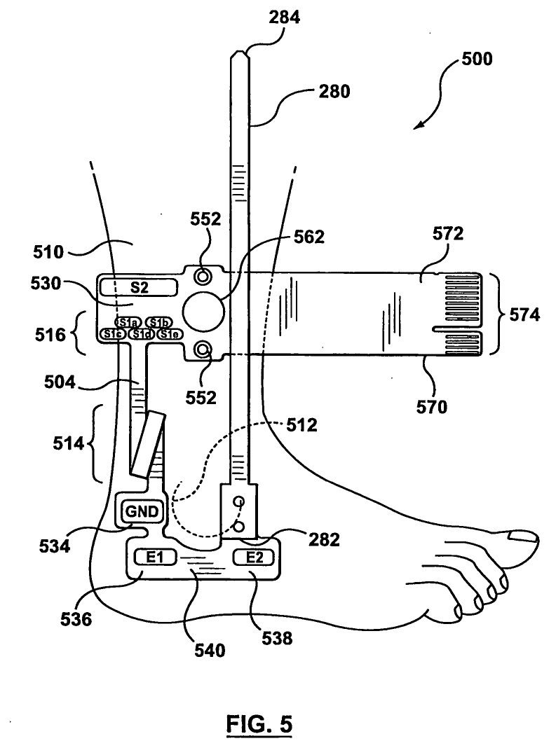

Referring now to FIG. 5, there is shown a further embodiment of a stimulus unit, designated by reference numeral 500. Stimulus unit 500 is intended for use in nerve conduction testing of the sural nerve in a human leg. Stimulus unit 500 has a base portion 530 for location over the sural nerve on a lower part of a right leg, as shown in FIG. 5.

Connected to base portion 530 is a connector limb 572 having a connector 570 on an end thereof and connector conductors 574 exposed within connector 570. Connector 570 is received in a socket 222 of coupling unit 220. Similar to base portion 230, base portion 530 has snap projections 552 for connecting to corresponding recesses in coupling unit 220.

Base portion 530 has a reference stimulation electrode S2 formed in the substrate and an array 516 of active stimulation electrodes (S1a, S1b, S1c, S1d, S1e) formed distally of S2 in the substrate. The array 516 is used to selectively provide stimuli to different locations within an area covered by the array 516.

The substrate of stimulus unit 500 further comprises a distally extending limb 504 connected to, and integrally formed with, base portion 530. Limb 504 has an extensible portion 514 formed therein for allowing adjustment of the distance between the sensing and stimulus electrodes to account for different leg sizes. A distal end portion 540 is formed at a distal end of limb 504 and comprises sensing electrodes E1, E2. A ground electrode GND is also formed in limb 504, intermediate distal end portion 540 and the extensible portion 514.

Distal end portion 540 has attachment portions 536, 538 for securing electrodes E1, E2 to the skin of the ankle just below, and on either side of, the lateral malleolus 512. Ground electrode GND is attached to the skin using an adhesive attachment portion 534.

Distance measurement strip 280 is connected at fixed end 282 to a part of distal end portion 540 adjacent attachment portion 538. Distance measurement strip 280 extends proximally toward base portion 530 so that free end 284 can be passed through slot 240 of coupling unit 220 for measurement of the distance between the sensing electrodes E1, E2 and the stimulation electrodes S2, S1a to S1e.

As shown in FIG. 5, opening 562 in base portion 530 is located between projecting connector parts 552. In such a configuration, the coupling unit 220 has a temperature sensor 260 positioned in between recessed connecting parts 250 to correspond with the configuration of base portion 530. Such a modified coupling unit 220 may also be used with the stimulus unit shown FIG. 4, with opening 262 being positioned in between projecting connector parts 252.

It should be noted that stimulus unit 500 is one specific embodiment of the more general embodiment of stimulus unit 130 described above. Thus, while stimulus unit 500 is of a different shape and configuration to that shown in FIG. 3, for example, it is formed in a similar manner, using similar materials and is used in a similar way.

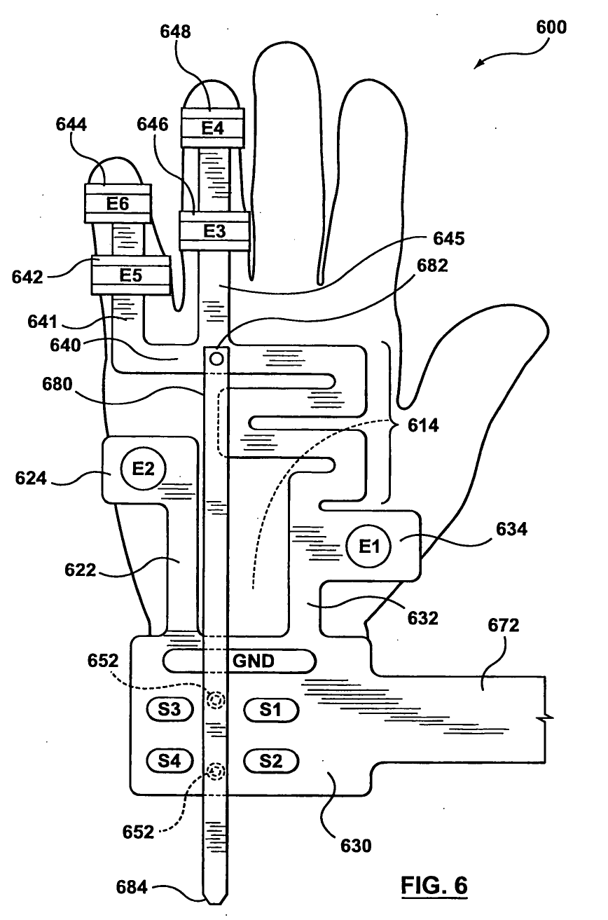

FIG. 6 shows a further embodiment of a stimulus unit, designated by reference numeral 600. Stimulus unit 600 is formed of similar materials and operates in a similar way to the stimulus unit embodiments shown in FIGS. 2 to 5, except that it has a different electrode configuration and a modified extensible portion 614.

Stimulus unit 600 has a first limb 632 and a second limb 622, both of which extend distally from a distal edge or part of base portion 630. First limb 632 has a first sensing electrode E1 formed in a part of the substrate that is positioned to generally overlie a thenar muscle. Electrode E1 is held on to the thenar area by an adhesive-backed attachment portion 634.

Extensible portion 614 is formed distally of attachment portion 634 in limb 632. Extensible portion 614, as shown in FIG. 6, is formed of a plurality of loops formed in the plane of the substrate in a snaking, s-shape. Distally of extensible portion 614, first limb 632 branches into a first branch 641 and a second branch 645. First branch 641 has sensing electrodes E5, E6 formed in attachment portions 642, 644, which attach the electrodes E5, E6 to appropriate locations on the fifth digit (little finger). The second branch 645 comprises sensing electrodes E3, E4 located in attachment portions 646, 648 for attaching the electrodes E3, E4 to appropriate locations on the fourth digit (ring finger). Sensing electrode pair E5, E6 can be used to sense evoked SNAP responses resulting from stimulation of the ulnar nerve by stimulation electrodes S3, S4 positioned over the ulnar nerve.

Sensing electrode pair E3, E4 can be used to sense evoked SNAP responses for both ulnar and median nerves, in response to stimulus from the ulnar stimulus pair S3, S4 or median stimulus pair S1, S2. Sensing electrode E1 is used to detect CMAP responses to stimulus from the median stimulating electrode pair S1, S2.

Second limb 622 has a sensing electrode E2 positioned toward a distal end of limb 622 and attached to a hypothenar area of the hand by adhesive attachment portion 624. Electrode E2 is positioned to sense evoked CMAP responses resulting from stimulus of the ulnar nerve by stimulation electrode pair S3, S4.

Stimulus electrodes S1 to S4, together with a ground electrode GND are formed in the substrate in base portion 630. The connecting projection parts 652 are also formed in base portion 630 for connecting to coupling unit 220, either as a purely mechanical connection or as electrically conductive connectors for supplying stimulus to the stimulus electrodes S1 to S4.

A connector limb 672 extends laterally from base portion 630 and has a connector (not shown) on an end thereof for connecting to socket 222 of coupling unit 220 to provide the detected evoked signals back to the stimulus and data acquisition module 120.

As shown in FIG. 6, a distance measurement strip 680 is connected to first limb 632, at a connection portion 640 thereof. Fixed end 682 is connected to connection portion 640, while free end 684 of distance measurement strip 680, extends proximally toward base portion 630, for insertion into slot 640 of coupling unit 220, when coupling unit 220 is attached to base portion 630. As with other embodiments employing a distance measurement strip, distance measurement strip 680 has some form of indicia formed on or in the strip along at least part of its length for reading by a scanner 290 in coupling unit 220 or for comparison with an alignment marker on base portion 630.

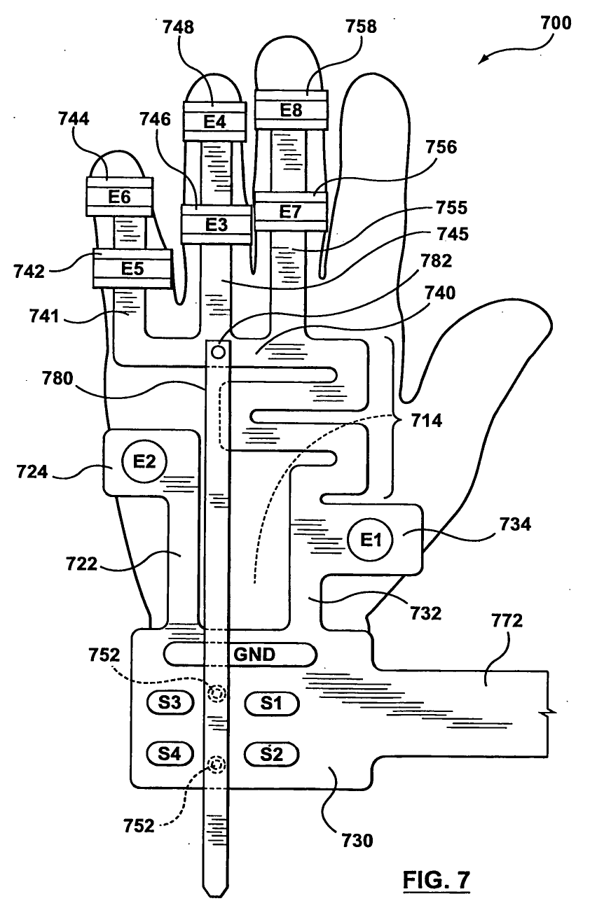

Referring now to FIG. 7, a schematic representation of a further embodiment of a stimulus unit is shown, designated by reference numeral 700. Stimulus unit 700 is identical to stimulus unit 600, except that the first limb 732 of stimulus unit 700 has three branches, rather than two. Stimulus unit 700 is formed of similar materials and operates in a similar way to the stimulus unit embodiments shown in FIGS. 2 to 5, except that it has a different electrode configuration and a modified extensible portion 714.

Stimulus unit 700 has a first limb 732 and a second limb 722, both of which extend distally from a distal edge or part of base portion 730. First limb 732 has a first sensing electrode E1 formed in a part of the substrate that is positioned to generally overlie a thenar muscle. Electrode E1 is held on to the thenar area by an adhesive-backed attachment portion 734.

Extensible portion 714 is formed distally of attachment portion 734 in limb 732. Extensible portion 714, as shown in FIG. 7, is formed of a plurality of loops formed in the plane of the substrate in a snaking, s-shape. Distally of extensible portion 714, first limb 732 branches into a first branch 741 and a second branch 745. First branch 741 has sensing electrodes E5, E6 formed in attachment portions 742, 744, which attach the electrodes E5, E6 to appropriate locations on the fifth digit (little finger). The second branch 745 comprises sensing electrodes E3, E4 located in attachment portions 646, 648 for attaching the electrodes E3, E4 to appropriate locations on the fourth digit (ring finger). Sensing electrode pair E5, E6 can be used to sense evoked SNAP responses resulting from stimulation of the ulnar nerve by stimulation electrodes S3, S4 positioned over the ulnar nerve. The third branch 755 has sensing electrodes E7, E8 located in attachment portions 756, 758 for positioning electrodes E7, E8 at appropriate locations around the third digit (middle finger).

Sensing electrode pair E3, E4 can be used to sense evoked SNAP responses for both ulnar and median nerves, in response to stimulus from the ulnar stimulus pair S3, S4 or median stimulus pair S1, S2. Sensing electrode E1 is used to detect CMAP responses to stimulus from the median stimulating electrode pair S1, S2.

Second limb 722 has a sensing electrode E2 positioned toward a distal end of limb 722 and attached to a hypothenar area of the hand by adhesive attachment portion 724. Electrode E2 is positioned to sense evoked CMAP responses resulting from stimulus of the ulnar nerve by stimulation electrode pair S3, S4.

Stimulus electrodes S1 to S4, together with a ground electrode GND are formed in the substrate in base portion 730. The connecting projection parts 752 are also formed in base portion 730 for connecting to coupling unit 220, either as a purely mechanical connection or as electrically conductive connectors for supplying stimulus to the stimulus electrodes S1 to S4.

A connector limb 772 extends laterally from base portion 730 and has a connector (not shown) on an end thereof for connecting to socket 222 of coupling unit 220 to provide the detected evoked signals back to the stimulus and data acquisition module 120.

As shown in FIG. 7, a distance measurement strip 780 is connected to first limb 732, at a connection portion 740 thereof. Fixed end 782 is connected to connection portion 740, while free end 784 of distance measurement strip 780, extends proximally toward base portion 730, for insertion into slot 740 of coupling unit 220, when coupling unit 220 is attached to base portion 730. As with other embodiments employing a distance measurement strip, distance measurement strip 780 has some form of indicia formed on or in the strip along at least part of its length for reading by a scanner 290 in coupling unit 220 or for comparison with an alignment marker on base portion 730.

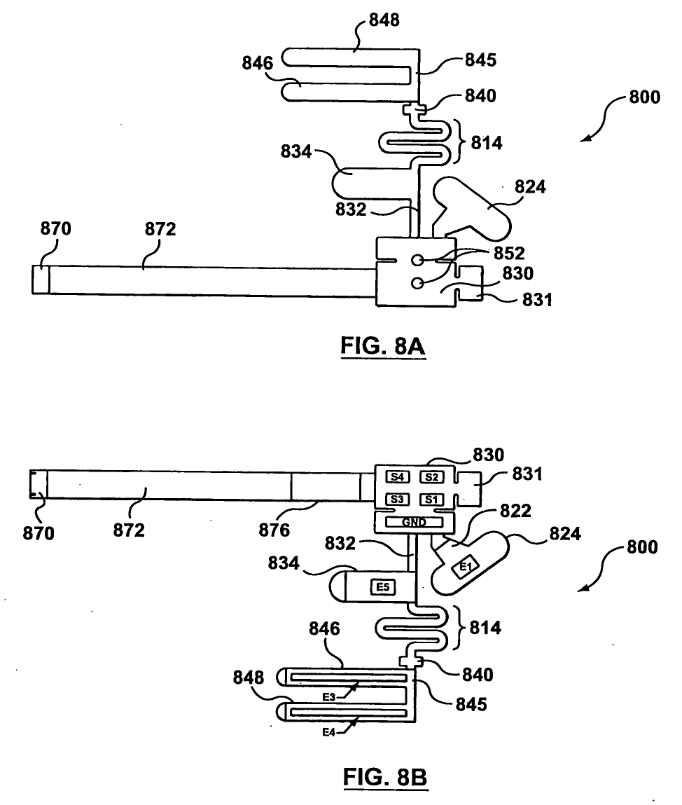

FIGS. 8A and 8B show respective plan and bottom views of a further embodiment of a stimulus unit, designated by a reference numeral 800. Stimulus unit 800 is for placement on a right hand for stimulation of the median and ulnar nerves in a manner similar to that described in relation to FIG. 4. Stimulus unit 800 has a first limb 832 extending distally from a generally central part of a distal edge or portion of base portion 830. Stimulus unit 800 also has a second limb 822 extending distally toward a thenar area, when placed on a hand. Second limb 822 has an adhesive attachment portion 824 for attaching a sensing electrode E1 over a part of the thenar area. Stimulus unit 800 is similar to stimulus unit embodiments 600 and 700, in that it has two distally extending limbs, one of which has only a CMAP sensing electrode and the other of which has both CMAP and SNAP sensing electrodes separated by an extensible portion 814.

A connection portion 840 is formed at a distal end of extensible portion 814, but proximally of a first branch 845 which supports sensing electrodes E3, E4 formed at adhesive attachment portions 846, 848 for attaching electrodes E3, E4 to a fourth digit (ring finger). Connection portion 840 is of a sufficient dimension to enable attachment of a fixed end of a distance measurement strip, such as anyone of those shown and described in relation to previous embodiments or in relation to FIGS. 11, 12 or 13.

Extensible portion 814 is formed so as to have a plurality of loop portions extending in a snaking pattern in the same plane as that of the rest of the substrate. Proximal of extensible portion 814 on first limb 832 but distal of base portion 830, a sensing electrode E5 is located, within adhesive attachment portion 834. Sensing electrode E5 is positioned so as to be able to overlie a hypothenar area of the right hand.

As shown in FIG. 8B, an adhesive attachment portion 876 is provided along a part of connector limb 872, somewhat adjacent base portion 830, for assisting secure attachment of stimulus unit 800 to the wrist. Further, an additional adhesive attachment portion 831 is provided on opposite side of base portion 830 to that of attachment portion 876. Attachment portion 831 serves to provide additional surface area for adhesive attachment of stimulus unit 800 to the wrist area.

Connector limb 872, is formed of a greater length than other embodiments, as it is designed to wrap around the wrist so that connector 870 can connect to socket 222 and coupling unit 220 from the right side (as viewed in plan view). As with other embodiments described herein, stimulus unit 800 has projecting connector parts 852 on base portion 830 for connecting to coupling unit 220. Specifically, the stimulating electrodes make up one adhesive section. At least one sensing electrode makes up another separate adhesive section and the separate adhesive sections are connected by and extensible non-adhesive section. In this way the stimulating and sensing electrodes sections can be place a variable distance apart to accommodate different sizes and varying anatomy.

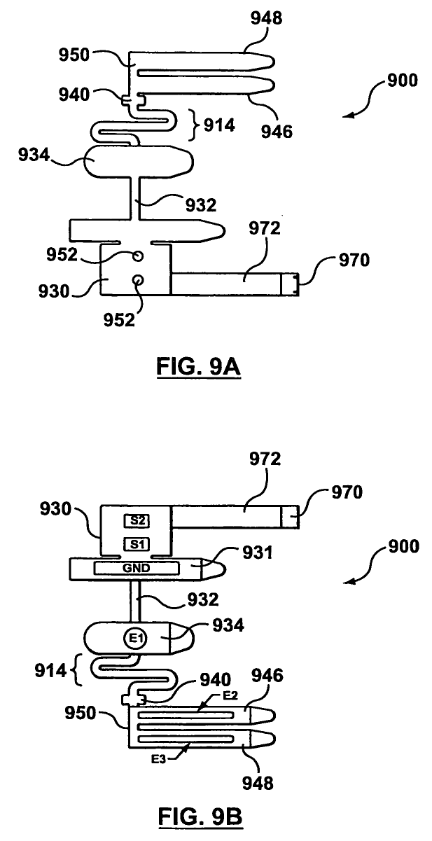

Referring now to FIGS. 9A and 9B, a further stimulus unit embodiment is shown, designated by reference numeral 900. Stimulus unit 900 is similar to stimulus unit 800, except that it is only designed for testing of the ulnar nerve. Consequently, stimulus unit 900 only has a single pair of stimulating electrodes S1, S2 positioned on base portion 930. Stimulus unit 900 has a single limb 932 projecting distally from a ground electrode GND and adhesive attachment portion 931 formed immediately distally of base portion 930.

Stimulus unit 900 has an extensible portion 914 formed in limb 932, intermediate a first sensing electrode E1 for overlying a hypothenar area and distal sensing electrodes E2, E3 for attachment to a fifth digit (little finger). Sensing electrode E1 is attached to the hypothenar area by adhesive attachment portion 934, while sensing electrodes E2, E3 are attached to the fifth digit (little finger) by adhesive attachment portions 946, 948 respectively.

Stimulus unit 900 has a connection portion 940 for receiving in a connecting fashion the fixed end of a distance measurement strip, such as any one of those shown and described in relation to other embodiments or as shown and described in relation to FIGS. 11 to 13. Connection portion 940 is formed in limb 932 distal of extensible portion 914 but proximal of a branch 950 from which electrodes E2, E3 extend laterally.

Like stimulus unit 800, stimulus unit 900 has projecting connection parts 952 for connecting the base portion 930 to coupling unit 220. Further, a connector limb 972 extends from base portion 930 and has a connector 970 on an end thereof for receipt in socket 222 of coupling unit 220.

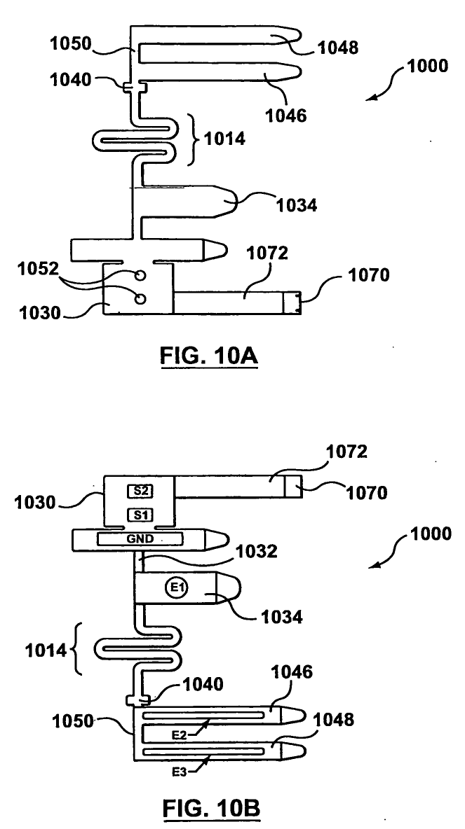

Referring now to FIGS. 10A and 10B, a further stimulus unit embodiment is shown, designated by reference numeral 1000. Stimulus unit 1000 is almost identical to stimulus unit 900, except that it is intended for stimulus of only the median nerve. Stimulus unit 1000 has a single pair of stimulating electrodes S1, S2 positioned on base portion 1030. Stimulus unit 1000 has with a single limb 1032 projecting distally from a ground electrode GND and adhesive attachment portion 1031 formed immediately distally of base portion 1030.

Stimulus unit 1000 has an extensible portion 1014 formed in limb 1032, intermediate a first sensing electrode E1 for overlying a thenar area and distal sensing electrodes E2, E3 for attachment to a third digit (middle finger) or optionally the fourth digit (ring finger). Sensing electrode E1 is attached to the thenar area by adhesive attachment portion 1034, while sensing electrodes E2, E3 are attached to the third or fourth digit (middle or ring finger) by adhesive attachment portions 1046, 1048 respectively.

Stimulus unit 1000 has a connection portion 1040 for receiving in a connecting fashion the fixed end of a distance measurement strip, such as any one of those shown and described in relation to other embodiments or as shown and described in relation to FIGS. 11 to 13. Connection portion 1040 is formed in limb 1032 distal of extensible portion 1014 but proximal of a branch 1050 from which electrodes E2, E3 extend laterally.

Turning now to FIG. 11, there is shown a representation of one embodiment of a distance measurement strip, designated by reference numeral 1180. Distance measurement strip 1180 has a fixed end 1182 for fixation to connection portion 1040, 940, 840, 740, 640, 540 or another point adjacent to the distal sensing electrodes. On an opposite end of distance measurement strip 1180 is a free end 1184 for insertion into, and passage through, slot 240 of coupling unit 220.

Intermediate fixed end 1182 and free end 1184, quadrature indicia 1186 are printed or otherwise placed on a surface of distance measurement strip 1180 for scanning by scanners 290. The quadrature pattern of indicia 1186 is used by control module 110 to determine the relative amount of progress of distance measurement strip 1180 through slot 240, together with the known separations of other parts of stimulus unit 130 (or 500, 600, 700, 800, 900 or 1000) and the predetermined physical relationship of coupling unit 220 to the base portion of the stimulus unit.

In addition to the distance measurement indicia 1186, identifying indicia 1188 is provided on a part of distance measurement strip 1180 toward free end 1184. This further indicia specifies a unique identifier of the stimulus unit, such as a serial number or other form of unique identifier for tracking the use of the stimulus unit to ensure that it was used once only. The identifying indicia 1188 may also indicate a type of the stimulus unit (e.g. right median, left sural) and/or a use-by date (because the conductive gel tends to dry over time). Unique identifier indicia 1188 may be encoded, for example, in the form of a barcode or other machine-readable code so that it can be read into stimulus and data acquisition module 120 via scanners 290 and subsequently recorded into memory 112.

Referring now to FIG. 12, there is shown a further embodiment of a distance measurement strip, designated by reference numeral 1280. Distance measurement strip 1280 has a fixed end 1282 for use in the manner described above in relation to fixed end 1182. Similarly, an opposite free end 1284 is provided on the elongate strip.

Distance measurement strip 1280 has distance related indicia 1286 printed or otherwise placed on a portion thereof toward fixed end 1282, while indicia specifying a unique identifier is provided on distance measurement strip 1280 more toward free end 1284. Distance measurement related indicia 1286 and the identifier related indicia 1288 employ a gray scale for determining the distance or unique identifier. Calibration indicia 1289 are also provided proximate free end 1284 for calibration of the light intensity signals returned to scanner 290 from light impinging on the gray scale indicia.

Distance measurement indicia 1286 may comprise a strip of continuously darkening gradations corresponding to the distance of travel of distance measurement strip 1280 through slot 240. The light intensity signals thus returned by scanner 290 (only one scanner 290 is required for sensing gray scale intensity) may be interpreted by stimulus and data acquisition module 120 or processor 114 to determine the distance that corresponds to the gray scale position at which distance measurement strip 1280 comes to rest in front of scanner 290. Identification indicia 1288 may use gray blocks to encode a unique identifier.

Referring now to FIG. 13, there is shown a further embodiment of a distance measurement strip, designated by reference numeral 1380. Like the embodiments of FIGS. 11 and 12, distance measurement strip 1380 has a fixed end 1382 for connection to a distal part of stimulus unit 130 and an opposite free end 1384 for receipt in slot 240. Distance measurement indicia 1386 comprises a series of equally spaced, equal width bars of about two millimeters in width. Identification indicia 1388 may include a numeric identifier, such a serial number, together with an encoded version of the numeric identifier. The encoded identifier may be encoded in the form of a barcode or gray scale, for example.

For each of the distance measurement strip embodiments shown in FIGS. 11 to 13, the length of the elongate strip will depend on the form and configuration of the stimulus unit 130 and the position on the distally extending limb to which it is attached. However, embodiments of the distance measurement strip may have a length in the order of 20 to 40 centimeters or in the vicinity of 30 to 35 centimeters.

Other embodiments of the distance measurement strip may use indicia that can be sensed by a scanner 290 that is not purely optical in nature. Further, according to alternative embodiments, the identification indicia 1188, 1288 or 1388 may be formed on a part of stimulus unit 130 other than the distance measurement strip.

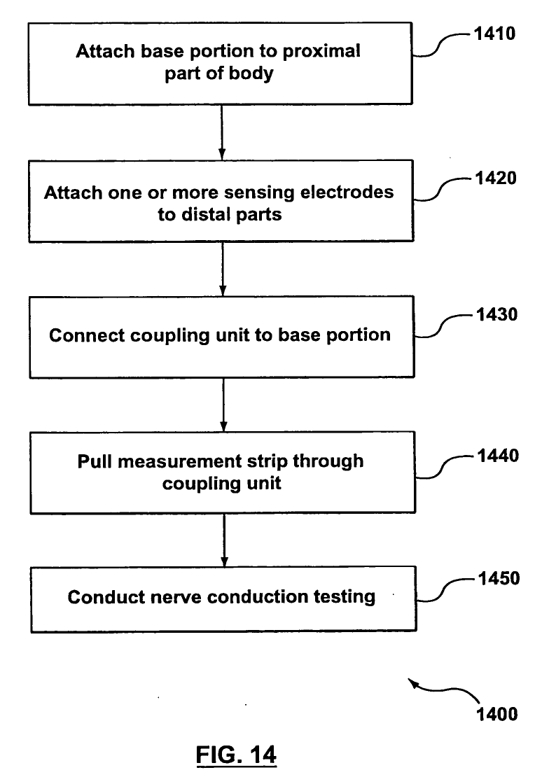

Referring now to FIG. 14, there is shown a flow chart of a method of nerve conduction testing that involves measuring the separation between at least one distal electrode on a limb of the stimulus unit 130 (or 500, 600, 700, 800, 900 or 1000) and one of the proximal stimulation electrodes on the base portion. The method is designated by reference numeral 1400 and begins at step 1410, at which the base portion 230, 530, 630, 730, 830, 930,1030 is attached to a proximal part of the body, such as the forearm proximal of the wrist crease, or a lower leg proximal of the ankle. The base portion is attached in the desired position by peeling a backing sheet from the stimulus unit 130, 500, 600, 700, 800, 900, 1000 from the base portion so that the adhesive attachment portion on the underside of the base portion is exposed and can be adhered to the body.

At step 1420, the distal sensing electrodes are attached in the appropriate locations using the adhesive attachment portions surrounding each sensing electrode, with the backing sheets removed. In step 1430, coupling unit 220 is connected to the base portion using the corresponding connecting parts 250 on coupling unit 220 and connecting parts 252, 552, 652, 752, 852, 952, 1052 on the base portion of the stimulus unit.

At step 1440, the distance measurement strip 280, 680, 780, 1180, 1280, 1380 is inserted into slot 240 of coupling unit 220 and pulled and/or pushed therethrough so that the indicia on the distance measurement strip is read by one or more scanners 290. The signals generated by scanners 290 in response to the passage or final rest position of the distance measurement strip are transmitted from coupling unit 220 to a controller within stimulus and data acquisition module 120 and then onto processor 114 for processing to determine the separation of the stimulus and sensing electrodes.

At step 1450, the nerve conduction testing is carried out using the stimulus and sensing electrodes on stimulus unit 130, 500, 600, 700, 800, 900, 1000 and taking into account the determined separation of the stimulus and sensing electrodes as necessary.

It should be noted that, while the sensing electrodes are generally described herein as being distally positioned and the stimulation electrodes are described as being more proximally positioned, these positions represent nerve conduction testing in an antidromic orientation. It should be understood, however, that the relative functions of the sensing and stimulating electrodes may be reversed to an orthodromic orientation. In an orthodromic orientation, the stimulus may be applied at the fingers and/or thenar and/or hypothenar areas and the evoked response sensing may occur at the wrist, for example.

Referring now to FIG. 15, there is shown an example side cross-section of a base portion of a substrate according to an illustrative embodiment of a stimulus unit. The illustrative substrate is designated by reference numeral 1500 and is shown prior to application to a body part.

Substrate 1500 has a base layer 1510, which forms the top (or upper or outer) layer facing away from the body part. This base layer 1510 is formed of medical grade polyester or a similar material and has sufficient rigidity to form the base for flexible circuitry and enable subsequent conductive and insulative layers to be formed thereon, while having sufficient flexibility to enable the entire substrate 1500 to bend to generally conform to the shape of the body part to which it is to be affixed.

Electrodes 1520 are formed on base layer 1510, either directly or on a thin priming or separation layer (not shown) coating the underside of base layer 1510. Electrodes 1520 are electrically coupled to external connectors via conductors 1530 in the form of flexible circuit tracings formed on base layer 1510. As with electrodes 1520, conductors 1530 may be directly formed on base layer 1510 or may be separated therefrom by a priming or separation layer.

Portions of substrate 1500 that are not to be exposed to the body part (such as conductors 1520) are covered by an insulation layer 1535. This insulation layer 1535 covers conductors 1530 for electrodes 1520, which in the example cross-section are stimulating electrodes. Electrodes 1520 have a layer of conductive gel 1540 formed around them for facilitating conduction between electrodes 1520 and the skin of the body part on which the substrate 1500 is positioned.

For portions of substrate 1500 that are not covered by conductive gel 1540, but that surround the electrodes 1520 and conductive gel 1540, a double-sided adhesive layer 1550 is formed over the insulation layer 1535. Adhesive layer 1550 may be a foam (or other) material impregnated or coated with one or more adhesive substances or it may be a layer of the adhesive substance itself.

The adhesive layer 1550 and conductive gel 1540 is covered by a protective backing sheet or layer 1560 so that the adhesive and conductive qualities of the adhesive layer 1550 and conductive gel 1540 are preserved until application of substrate 1500 to the body part. The total thickness of substrate 1500 may be in the order of 0.7 to 1.5 millimeters, approximately.

The embodiment shown in FIG. 15 is not to scale, is for purposes of illustration only and some variations or modifications may be made, depending on the specific requirements of the stimulus unit embodiment and methods of forming it. Although the entire stimulus unit cross-section shown in FIG. 15 is described as the substrate and designated by reference numeral 1500, base layer 1510 may also be considered to be (or be part of a substrate, with electrodes 1520 and conductors 1530 being formed on the substrate.

While the stimulus unit embodiments shown and described herein generally show a unitary substrate including one or more limbs and a base portion, each of the areas or portions of the stimulus unit having sensing or stimulation electrodes may be formed on a separate substrate. For example, distal sensing electrodes positioned around a finger may be formed on a substrate distinct from the substrate on which the proximal stimulation electrodes are formed. In such embodiments of the stimulus unit, as conductors 1530 cannot be formed to cross between substrates, the separate substrates must be either electrically coupled to each other (for example, by connectors) or have separate connectors for interfacing with coupling unit 220. Such embodiments may be useful where, for example, the extensible portion in one of the limbs is formed as a strain gauge or other electromechanical sensor to indicate the degree of extension of the limb and thereby provide a measurement of the separation of the separate substrates and their respective electrodes. Such embodiments therefore do not require a distance measurement strip.

Reference herein to a limb is not intended to include a reference to a human limb, such as an arm or leg. Rather, it is a reference to a part of a stimulus unit embodiment.

1. A device for use in measuring neuromuscular function, comprising:
a substrate, the substrate having a base portion and at least one limb coupled to the base portion;
at least two stimulation electrodes operably associated with the substrate for providing a stimulus to a body part;
at least two sensing electrodes operably associated with the substrate for sensing an electrical potential in the body part, wherein the at least two sensing electrodes are spaced from the at least two stimulation electrodes; and
a distance measurement member coupled to one of the base portion and the at least one limb for use in indicating separation of the at least two stimulation electrodes and the at least two sensing electrodes.

2. The device of claim 1, wherein the distance measurement member comprises indicia for indicating separation of the at least two stimulation electrodes and the at least two sensing electrodes.

3. The device of claim 2, wherein the indicia is selected from the group consisting of: magnetic indicia; electrical indicia; optical indicia;
electromechanical indicia; and mechanical indicia.

4. The device of claim 1, wherein the distance measurement member comprises an elongate strip.

5. The device of claim 1, wherein the substrate is flexible to conform to a shape of the body part when positioned over the body part.

6. The device of claim 1, wherein the body part comprises a hand and wrist.

7. The device of claim 1, wherein the body part comprises a leg and ankle.

8. The device of claim 1, wherein the stimulation and sensing electrodes each have a layer of conductive gel disposed on the stimulation and sensing electrodes for contact with the body part.

9. The device of claim 8, wherein the stimulation and sensing electrodes are covered by a protective material, and wherein, in use of the device, the protective material is removed before placement of the stimulation and sensing electrodes on the body part.

10. The device of claim 1, wherein the at least two sensing electrodes are positioned distal to the at least two stimulating electrodes along the body part.

11. The device of claim 1, wherein the at least two sensing electrodes are positioned proximal to the at least two stimulating electrodes along the body part.

12. The device of claim 1, wherein the at least two sensing electrodes comprise a first electrode pair, wherein one of the sensing electrodes in the pair is an active electrode, and the other of the sensing electrodes in the pair is a reference electrode.

13. The device of claim 2, further comprising a scanner for reading the indicia on the distance measurement member.

14. The device of claim 1, wherein one of the substrate and the distance measurement member comprises a unique identifier of the device;

15. The device of claim 14, wherein the unique identifier is encoded on one of the distance measurement member and the substrate.

16. The device of claim 15, wherein the unique identifier is machine-readable.

17. The device of claim 1, further comprising a coupling unit for electrically coupling a current stimulation controller to the at least two stimulating electrodes.

18. The device of claim 17, wherein the coupling unit is supportingly connectable to the base portion of the substrate.

19. The device of claim 18, wherein the coupling unit is connectable to the base portion by conductive connectors.

20. The device of claim 17, wherein the coupling unit comprises a temperature sensor.

21. The device of claim 20, wherein the temperature sensor is an infrared optical sensor.

22. The device of claim 17, wherein the substrate comprises at least one flexible conductor extending through the at least one limb for carrying current from the at least two sensing electrodes to the coupling unit.

23. The device of claim 22, wherein the device further comprises a connector limb having a connector on an end of the connector limb, the connector being connectable to the coupling unit.

24. The device of claim 23, wherein the connector on the connector limb is an output connector for providing at least one output of the at least two sensing electrodes.

25. The device of claim 19, wherein the conductive connectors are coupled to the at least two stimulation electrodes.

26. The device of claim 1, wherein the at least one limb comprises two limbs, each limb comprising at least one sensing electrode.

27. The device of claim 26, wherein each limb extends to a different designated area of the body part.

28. The device of claim 27, wherein the designated area is selected from the group consisting of: thenar, hypothenar, second digit, third digit, fourth digit and fifth digit.

29. The device of claim 17, wherein the coupling unit comprises a connector for coupling to conductors extending along the substrate.

30. The device of claim 17, wherein the coupling unit comprises a distance measurement sensor for cooperating with the distance measurement member to indicate the separation of the at least two sensing electrodes and the at least two stimulation electrodes.

31. The device of claim 30, wherein the distance measurement member comprises an elongate strip and the coupling unit has an opening for receiving the elongate strip.

32. The device of claim 31, wherein the distance measurement sensor is housed within the coupling unit and positioned to cooperate with the distance measurement member to indicate the separation when the elongate strip is received in the opening.

33. The device of claim 32, wherein the opening extends through the coupling unit and at least part of the elongate strip may be drawn through the opening.

34. The device of claim 1, wherein the at least one limb comprises a plurality of branches, each branch comprising at least one sensing electrode.

35. The device of claim 34, wherein each branch comprises two sensing electrodes.

36. The device of claim 1, wherein the at least one limb comprises an extensible portion intermediate the base portion and a distal end of the at least one limb.

37. The device of claim 36, where the extensible portion is disposed intermediate two of the at least two sensing electrodes.

38. The device of claim 36, wherein the extensible portion comprises at least one loop formed in the at least one limb.

39. The device of claim 38, wherein the elongate member is coupled to the at least one limb intermediate the extensible portion and the distal end of the at least one limb.

40. The device of claim 9, wherein the sensing and stimulation electrodes each have an adhesive material or substance disposed around the respective electrode.

41. The device of claim 1, wherein the substrate is a unitary substrate.

42. A stimulus unit for use in measuring neuromuscular function, comprising:
a substrate, the substrate having a base portion and at least one limb coupled to the base portion;
at least two stimulation electrodes operably associated with the substrate for providing a stimulus to a body part; and
at least two sensing electrodes operably associated with the substrate for sensing an electrical potential of the body part, wherein the at least two sensing electrodes are spaced from the at least two stimulation electrodes and wherein the at least one limb has at least one extensible section for permitting adjustment of the spacing between the at least two sensing electrodes and the at least two stimulation electrodes.

43. A device for use in measuring neuromuscular function, comprising:
a substrate, the substrate having a base portion and at least one limb coupled to the base portion;
at least two stimulation electrodes operably associated with the substrate for providing a stimulus to a body part;
at least two sensing electrodes operably associated with the substrate for sensing an electrical potential in the body part in response to the stimulus, wherein the at least two sensing electrodes are spaced from the at least two stimulation electrodes; and
distance measurement means fixed relative to the substrate for measuring separation of the at least two stimulation electrodes and the at least two sensing electrodes.

44. A stimulus unit for use in measuring neuromuscular function, comprising:
a substrate, the substrate having a base portion and two limbs coupled to the base portion;
at least two stimulation electrodes operably associated with the base portion for providing a stimulus to a body part; and
at least one sensing electrode operably associated with each limb for sensing an electrical potential in the body part in response to the stimulus, wherein the at least two stimulation electrodes are spaced from the sensing electrodes.

45. A method of measuring a separation between at least one distal electrode and at least one proximal electrode, the at least one distal electrode having an elongate member fixed relative to the at least one distal electrode, indicia being formed on or in the elongate member, the method comprising:
affixing the at least one proximal electrode to a proximal part of a body;
affixing the at least one distal electrode to a distal part of the body;
securing a scanner relative to the at least one proximal electrode; and
drawing a free end of the elongate member past the scanner to expose the indicia to the scanner and thereby measure the separation between the at least one distal electrode and the at least one proximal electrode.

46. A method of measuring a separation between at least one distal electrode and at least one proximal electrode, the at least one distal electrode being coupled to the at least one proximal electrode using an extensible electromechanical distance measurement sensor, the method comprising:
affixing the at least one proximal electrode to a proximal part of a body;
extending the at least one distal electrode relative to the at least one proximal electrode by extending the electromechanical distance measurement sensor; and
determining the separation based on an output of the extended electromechanically distance measurement.
